(19) United States
(12) Patent Application Publication
(10) Pub. No.: US 2010/0010302 A1
(43) Pub. Date: Jan. 14, 2010

(54) ENDOSCOPIC REFLECTOR

(75) Inventor: Ron Hadani, Cresskill, NJ (US)

Correspondence Address: MARTIN D. MOYNIHAN d/b/a PRTSI, INC., P.O. BOX 16446, ARLINGTON, VA, 22215, US

(73) Assignee: Vision-Sciences Inc., Orangeburg, NY (US)

(21) Appl. No.: 12/528,476

(22) PCT Filed: Feb. 26, 2008
(86) PCT No.: PCT/IL08/00241

§ 371 Date: Aug. 25, 2009

Related U.S. Application Data
(60) Provisional application No. 60/903,288, filed on Feb. 26, 2007.

(51) Int. Cl. A61B 1/04; A61B 1/06

(52) U.S. Cl. 600/109; 600/173

(57) ABSTRACT

A reflector for an endoscopic tool, which is made, for example of a framework or a balloon and reflects an image proximal to the tip to an imager at or near a tip of the endoscope. In some embodiments, the reflector distorts the image, which may be corrected using software.

### RELATED APPLICATIONS

This application claims the benefit under 119(e) of U.S. Provisional Application No. 60/903,288 filed 26 Feb. 2008, the disclosure of which is incorporated herein by reference.

### FIELD OF THE INVENTION

The present invention, in some embodiments thereof relates to endoscopes and particularly to methods of acquiring images by endoscopes.

### BACKGROUND OF THE INVENTION

Endoscopes are used to view internal tissue of humans, and to access body tissue for taking biopsy samples, delivery of therapeutic means and/or introduction of fluids. An endoscope generally either includes a camera at its distal end or has a fiber optic image bundle which leads images from a distal end of the endoscope to a proximal end thereof.

U.S. patent publication 2003/0167007 to Belson, the disclosure of which is incorporated herein by reference, describes a colonscope with a spectroscopic examination unit extending therefrom. The spectroscopic examination unit is rotatable relative to the colonscope.

U.S. Pat. Nos. 6,736,773 and 7,004,900 to Wendlandt et al., patented May 18, 2004 and Feb. 28, 2006, respectively, the disclosures of which are incorporated herein by reference, describe an endoscope with a vision head mounted on an extension arm for moving the vision head away from the endoscope. The vision head may be a parabolic mirror for reflecting images from behind the distal end of the endoscope to a vision chip on the endoscope.

While the vision head is useful in acquiring images from a large span of regions, it may interfere in performing other tasks of the endoscope.

U.S. Pat. No. 6,997,924 to Schwartz et al., patented Feb. 14, 2006, the disclosure of which is incorporated herein by reference, describes a catheter having an optical assembly for emission of laser light energy. An anchoring balloon is expanded to position a mirror near the ostium such that light energy from the laser optical assembly is reflected and directed circumferentially around the ostium.

German patent DE 297 16 512, the disclosure of which is incorporated herein by reference, describes a mirror head having a rotatable mirror which is introduced through a working channel of an endoscope and is used to project light for spectral analysis at walls of a body lumen in which the endoscope is located.

### SUMMARY OF THE INVENTION

An aspect of some embodiments of the present invention relates to using a collapsible reflector head to direct images towards an image capturing unit on a distal end of an endoscope.

Optionally, the reflector head is positioned distal from the image capturing unit. In some embodiments of the invention, when it is desired to acquire images in a backward direction, the reflector head is expanded, while when it is desired to acquire images in a forward direction, the reflector head is collapsed. In other embodiments of the invention, the reflector head is used to direct a view toward a side looking or backward looking image capturing unit.

In some embodiments of the invention, the collapsible reflector head comprises a balloon made of a reflective material, having a reflective coating and/or having a mirror mounted thereon.

Alternatively or additionally, the collapsible reflector head comprises a foldable unit, for example in a manner similar to an umbrella. Optionally, the collapsible reflector head unfolds symmetrically, expanding substantially equally in all directions. Alternatively, the reflector head unfolds asymmetrically, expanding in one direction more than in another direction or expanding only in a single direction. In some embodiments of the invention, the reflector head is mounted on a foldable frame. Optionally, the foldable frame is formed of rigid segments which fold at one or more axes. In some embodiments of the invention, the foldable frame includes at least three, at least four, at least five or more folding points. Alternatively, for simplicity, the foldable frame includes only two or even only a single folding point.

Further alternatively or additionally, the collapsible reflector head comprises a deflectable head which in a collapsed state is located along an axis of the endoscope, having a small cross sectional area, and in an expanded state deflects away from the endoscope axis.

The collapsible reflector head optionally has a predetermined expanded shape having required optical characteristics, which do not distort images or alternatively distort them in a known manner which is correctable based on the known distortion. Alternatively or additionally, the collapsible reflector is expanded in a manner adapted to comply with the organ under endoscopic analysis and/or surrounding tissues. In some embodiments of the invention, correction software corrects distortions in the acquired image due to irregularities in the reflective surface of the collapsible reflector. Optionally, a degree of distortion is estimated using an indication of the layout of the reflector (e.g., inflation pressure). Optionally or alternatively, a calibration procedure is carried out, for example, by illuminating the reflector with a pattern whose distortion is imaged. Optionally or alternatively, a shape with a known pattern is imaged with the reflector in place, for example, using a calibration disc placed on the endoscope while outside the body, for calibration purposes. Calibration can be, for example, manual or automatic. Optionally, with image corrections entered by hand into a processing station.

In some embodiments of the invention, the collapsible reflector head is adapted to collapse in a manner which reduces its cross sectional area within the patient, by at least 20%, 30% or even 50%. Optionally, in the collapsed configuration, the reflector head can pass through a working channel, or other channel of an endoscope, so that it can be removed entirely from the patient and make room for other endoscopic tools, while the endoscope remains in the patient.

An aspect of some embodiments of the present invention relates to using a miniature reflector to direct images toward an image capturing unit on a distal end of an endoscope. A maximal size of the miniature reflector in either a collapsed or non-collapsed state is small enough to pass through a working channel of an endoscope. Alternatively or additionally, the maximal size of the miniature reflector is less than 50% of the cross-sectional area of the insertion tube of an endoscope. For example, the endoscope, can have a typical diameter of, for example, 15 mm, 10 mm, 7 mm, 5 mm, 2 mm or smaller or intermediate diameters, at a distal region thereof.

An aspect of some embodiments of the present invention relates to using a concave reflector, having a narrow view, to direct images toward an image capturing unit on a distal end of an endoscope.

There is provided in accordance with an exemplary embodiment of the invention, an endoscopic system, comprising:

an insertion tube, defining at least one channel along its length;

an imaging unit on the insertion tube; and

a reflector adapted to direct a view at the imaging unit and adapted to pass through the at least one channel.

In an exemplary embodiment of the invention, the reflector is mounted on a collapsible frame having an open state in which the reflector cannot pass through the at least one channel and a collapsed state in which the reflector can pass through the at least one channel. Optionally or alternatively, the reflector is mounted on a deflectable handle, deflected between a state in which the reflector cannot pass through the channel and a state in which the reflector can pass through the channel. Optionally or alternatively, the reflector is mounted on a frame including a plurality of arms having a fixed relative orientation at one end and adapted to move relative to each other on another end. Optionally or alternatively, the reflector is a collapsible reflector having an open state in which it is larger than the cross-section of the channel and a collapsed state in which it can pass through the channel. Optionally, the reflector is adapted to move a plurality of times between the open and collapsed state. Optionally or alternatively, the reflector is adapted to be folded in changing between the open state and the collapsed state. Optionally or alternatively, the reflector is adapted to change an optical curvature thereof in changing between the collapsed state and the open state.

In an exemplary embodiment of the invention, the reflector is mounted on a balloon or is part of a balloon. Optionally, the reflector is in the form of a coating on a balloon. Optionally or alternatively, the balloon is adapted to be inflated to a state in which a reflective surface thereof is flat or piecewise flat. Optionally or alternatively, the balloon is reinforced by stiffening ribs. Optionally or alternatively, the balloon is adapted to have a predetermined shape when inflated.

In an exemplary embodiment of the invention, the reflector is adapted to have a parabolic or spherical surface over most of an area that directs light toward the imaging unit. Alternatively, the reflector is adapted to have a convex surface over most of its area directing light toward the imaging unit.

In an exemplary embodiment of the invention, the imaging unit is at the distal end of the insertion tube. Optionally or alternatively, the imaging unit comprises a camera mounted on the insertion tube.

In an exemplary embodiment of the invention, the system comprises a correction software which corrects images for distortions caused by the reflector.

In an exemplary embodiment of the invention, the reflector in an unexpanded form is adapted to pass through a channel having a diameter of less than 4 millimeters.

In an exemplary embodiment of the invention, the reflector is adapted to be positioned relative to the imaging unit such that it directs a view from behind the imaging unit at the imaging unit.

In an exemplary embodiment of the invention, the at least one channel comprises a working channel.

In an exemplary embodiment of the invention, the reflector is adapted to have a planar surface over most of an area of the reflector that is adapted to direct light toward the imaging unit.

There is also provided in accordance with an exemplary embodiment of the invention a method of viewing images within a patient, comprising:

inserting an endoscope into the patient;

providing a reflector along the endoscope;

positioning the reflector distal to an image acquiring unit of the endoscope; and

acquiring images of tissue of the patient reflected by the reflector.

In an exemplary embodiment of the invention, the method comprises uncollapsing the reflector after it is moved out of the distal end of a channel of the endoscope.

In an exemplary embodiment of the invention, the reflector is mounted on a collapsible frame and comprising uncollapsing the frame after the reflector is moved out of the distal end of a channel of the endoscope.

In an exemplary embodiment of the invention, the reflector is mounted on a handle and comprising deflecting the handle after the reflector is moved out of the distal end of a channel of the endoscope.

In an exemplary embodiment of the invention, the method comprises moving the reflector back into a channel of the endoscope after acquiring images. Optionally or alternatively, the method comprises removing the reflector from the patient through the channel, while the endoscope is within the patient. Optionally or alternatively, providing a reflector along the endoscope comprises passing the reflector along an outer surface of the endoscope. Alternatively, providing a reflector along the endoscope comprises passing the reflector along a channel extending through the endoscope. Optionally, the reflector is elastically loaded in the channel, such that it expands upon exiting a distal end of the channel. Optionally, the method comprises retracting the reflector into the channel in a manner which causes the reflector to collapse due to the retraction.

In an exemplary embodiment of the invention, the method comprises changing a geometry of the reflector while it is within the patient.

There is also provided in accordance with an exemplary embodiment of the invention, an endoscopic system, comprising:

an insertion tube;

an imaging unit on the insertion tube; and

a reflector unit including a reflector and having a collapsed state, and an open state in which the reflector is adapted to direct a view from behind the imaging unit at the imaging unit. Optionally, the reflector unit comprises a balloon having a reflective surface. Optionally or alternatively, the reflector changes its geometry between the open and collapsed states of the reflector unit. Optionally or alternatively, the reflector does not change its structure between the open and collapsed states of the reflector unit.

There is also provided in accordance with an exemplary embodiment of the invention, an endoscopic image reflecting unit, comprising:

an elongate handle adapted to pass through a channel of an endoscope; and

a collapsible reflector head carrying a reflector having a collapsed state in which it is adapted to pass through a channel of an endoscope, and an open state in which it is suitable to direct images toward an imaging unit at sufficient quality for imaging. Optionally, the collapsible reflector head comprises a distal part of the handle adapted to be bent into a state in which the reflector is at an angle of at least 45 degrees with an axial axis of the handle and to be bent into an axial state in which a length of the reflector substantially coincides with the axial axis of the handle. Optionally, the collapsible reflector head has a single rest state, and wherein in the single rest state the reflector is at an angle of at least 45 degrees with an axial axis of the handle.

### BRIEF DESCRIPTION OF THE DRAWINGS

Exemplary non-limiting embodiments of the invention will be described with reference to the following description of the embodiments, in conjunction with the figures. Identical structures, elements or parts which appear in more than one figure are preferably labeled with the same or similar number in all the figures in which they appear, and in which:

FIG. 1 is a schematic illustration of an endoscope system, in accordance with an exemplary embodiment of the present invention;

FIG. 2 is a schematic exploded cross section illustration of a distal end of an insertion tube, in accordance with an exemplary embodiment of the invention;

FIGS. 3A and 3B illustrate a reflector unit in a collapsed and open state, respectively, in accordance with an exemplary embodiment of the invention;

FIG. 4 is a schematic illustration of a reflector unit, in accordance with another exemplary embodiment of the invention;

FIG. 5A is a schematic illustration of an insertion tube, with an expandable reflector, in accordance with an exemplary embodiment of the invention;

FIG. 5B is a schematic expanded view of the expandable reflector of FIG. 5A, in accordance with an exemplary embodiment of the invention;

FIGS. 6A-6C are schematic illustration of a reflector unit, in accordance with still another exemplary embodiment of the invention;

FIG. 6D is a schematic illustration of a reflector, in accordance with another exemplary embodiment of the invention;

FIG. 7 is a schematic illustration of a reflector unit, in accordance with still another exemplary embodiment of the invention;

FIG. 8 is a schematic cross-section illustration of a reflector unit, in accordance with an exemplary embodiment of the invention;

FIGS. 9A and 9B are cross-sectional views at 90° from each other, of a reflection unit, in accordance with another exemplary embodiment of the invention;

FIG. 9C illustrates an inner tube of the reflection unit of FIGS. 9A and 9B, in a rest state, in accordance with an exemplary embodiment of the invention; and

FIGS. 10A-10C are schematic illustrations of balloon reflector units, in accordance with still additional exemplary embodiments of the invention.

### DETAILED DESCRIPTION OF EXEMPLARY EMBODIMENTS

### General

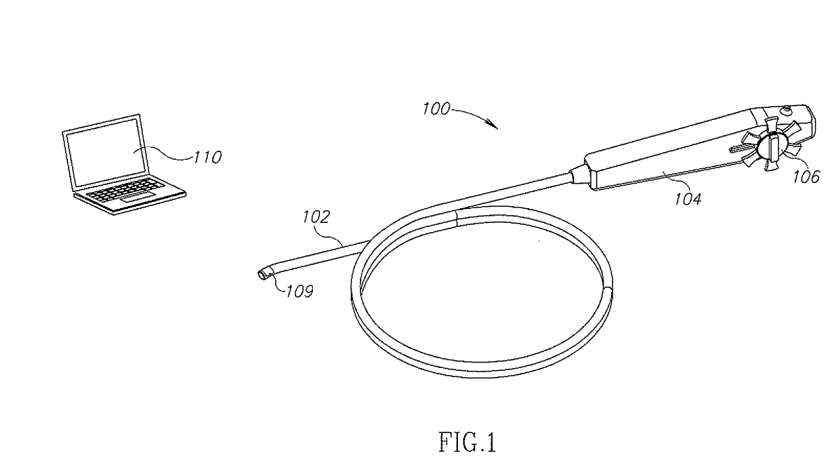

FIG. 1 is a schematic illustration of an endoscope system 100, in accordance with an exemplary embodiment of the present invention. System 100 optionally includes an elongate insertion tube 102 having a handle 104 carrying manipulation controls 106, an imaging head 109 and a control station 110, for example including a portable computer on which images acquired by imaging head 109 are displayed. Imaging head 109 is optionally directed in a fixed direction covering only a limited field of view. A reflection unit discussed in detail hereinbelow is used to direct images toward the imaging head. The reflector unit may be provided as an add-on to an endoscope not designed for use with a reflector unit or may be provided with an endoscope designed specifically for use with the reflector head.

In some embodiments of the invention, imaging unit 109 is designed to be used with the reflection unit, for example by being designed for viewing objects at farther optical paths and/or by having a higher resolution than imaging heads used only for forward viewing. In some embodiments of the invention, imaging unit 109 has an adjustable focal length which is adjusted according to whether the reflector unit is used or not.

Endoscope system 100 may be used for substantially any endoscopic procedure and the details (e.g., size, shape, elements included) of insertion tube 102, handle 104 and the other parts of system 100 may be in accordance with substantially any suitable endoscope known in the art and are optionally selected according to the specific task of the endoscope. In the following description an exemplary endoscope for examination of the intestine, is described.

Insertion tube 102 may be, for example, rigid, semi-rigid or rigid with a flexible bending section or flexible. Optionally, if flexible, the insertion tube is sufficiently flexible to allow it to make at least a 90° bend or 150° bend with a radius of less than 20 mm, or even less than 10 mm, for example so that it can negotiate the turns of the intestine or other torturous organ or orifice. Optionally, in one or more directions, insertion tube 102 can be manipulated to form a 180° bend or even at least a 270° bend over a distance of less than 30 millimeters. Possibly, insertion tube 102 allows different extents of bending in different directions. In an exemplary embodiment of the invention, insertion tube 102 is deflectable over 180°, or greater, in the up-down direction and over 160° in the left-right direction. Other deflection angles are possible as well, for other endoscope designs, for example, 50 degrees or 120 degrees.

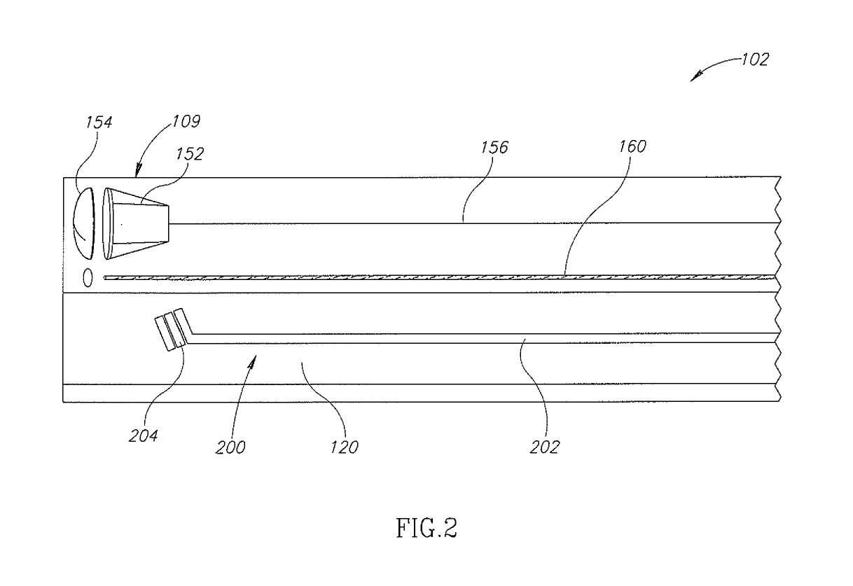

FIG. 2 is a schematic exploded cross section illustration of a distal end of insertion tube 102, in accordance with an exemplary embodiment of the invention. Insertion tube 102 includes imaging head 109 and one or more working channels 120. In some embodiments of the invention, imaging head 109 comprises a miniature camera 152 and a lens 154. Wires 156 optionally lead signals from camera 152 to a proximal end of insertion tube 102. Alternatively or additionally, a transmitter is used to wirelessly transmit images to control station 110. Possibly, imaging head 109 may include more than one camera and/or the camera may be movable and/or rotatable, in order to extend the range from which images may be acquired. Alternatively or additionally, imaging head 109 may include one or more optical fiber image bundles 160 which lead images along insertion tube 102.

Miniature camera 152 may include a CCD chip based camera, a CMOS chip based camera or any other camera known in the art. Imaging head 109 may be an integral part of insertion tube 102 with its elements being intermingled with other elements of insertion tube 102 or may be a separate unit with its elements being located in a distinct location within insertion tube 102. In some embodiments of the invention, imaging head 109 is separated from other parts of insertion tube 102 by walls. Possibly, imaging head 109 is removable from insertion tube 102, for example in accordance with any of the embodiments described in U.S. provisional patent application 60/806,162, filed Jun. 16, 2006, the disclosure of which is incorporated herein by reference.

A reflector unit 200 is optionally adapted to pass through working channel 120. Reflector unit 200 optionally includes a reflector head 204 (shown in FIG. 2 in a collapsed state) and a handle 202 which may be used to push reflector head 204 proximally and distally and/or to rotate reflector head 204. In some embodiments of the invention, handle 202 comprises a deflection mechanism which allows proper positioning of reflector head 204 above imaging head 109. Alternatively, handle 202 does not include a deflection mechanism, for simplicity, and the size of the open state of reflector head 204 is sufficient to allow reflector head 204 to perform its task.

In an exemplary embodiment of the invention, working channel 120 has a diameter of between 3-4 millimeters (e.g., 3.7 millimeters) and reflector unit 200, in a collapsed state, has a diameter smaller than that of the working channel. Other working channel diameters may be provided as well, for example, 1 mm, 1.8 mm, 2 mm 2.3 mm and intermediate and greater sizes. In some embodiments of the invention, in the collapsed state reflector head 204 may have a maximal diameter smaller than 3.5 millimeters, smaller than 3 millimeters or even smaller than 2 millimeters. It is noted, however, that the collapsing is not always performed in order to allow passage through working channel 120, but rather may be performed in order to limit the cross section area of reflector unit 200, so that it can be moved partially or entirely out of the view of camera 152 when desired. Alternatively or additionally, a collapsed reflector does not interfere with the movement of insertion tube 102, for example when reflector unit 200 does not have a shape adapted for fast movement within body cavities. In some embodiments of the invention, the cross section area of reflector unit 200, at least in the collapsed state, is less than 50%, 40% or even less than 30% of the cross section area of insertion tube 102.

Alternatively or additionally to passing through a working channel, reflector head 204 may pass through other channels, possibly a dedicated channel for reflector unit 200. Further alternatively or additionally, reflector unit 200 may be delivered before inserting insertion tube 102 into the patient. For example, reflector unit 200 may be inserted into the patient before insertion tube 102 and then serve as a guide wire for insertion tube 102. In some embodiments of the invention, reflector unit 200 may be delivered into the patient over insertion tube 102, for example in a manner in which insertion tube 102 serve as a monorail for reflector unit 200.

### Foldable Reflector

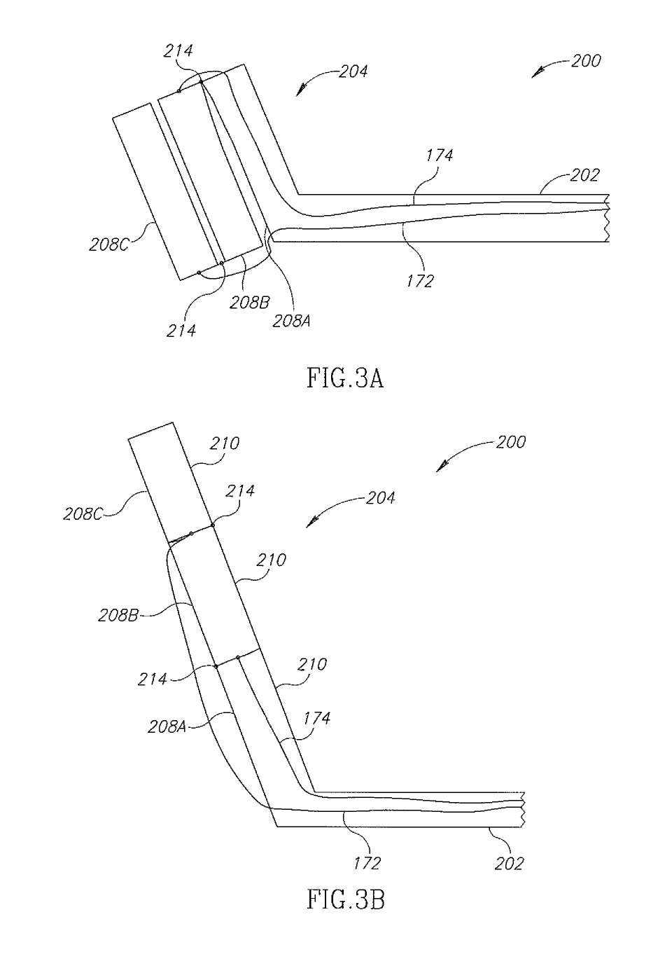

FIGS. 3A and 3B illustrate reflector unit 200, in accordance with an exemplary embodiment of the invention. Reflector head 204 comprises a plurality (e.g., three) of segments 208 (marked 208A, 208B and 208C), which are made of and/or coated with a reflective material on a surface 210. Reflective material may be any material that can reflect electromagnetic radiation with wavelengths in the range used for imaging, for example, aluminum and Mylar. Optionally, the reflective material is formed as a coating on the segment, for example, as a plasma deposited layer or a painted layer. Segments 208 are optionally connected to each other through pivots 214. Alternatively, segments 208 are formed from a single strip with predefined living hinges, formed on the strip. In a folded state, shown in FIG. 3A, reflector unit 200 is small enough to pass through working channel 120 (FIG. 2). In an unfolded state, shown in FIG. 3B, surfaces 210 of segments 208 define a large mirror, which can be used to direct a view from behind imaging head 109 and/or from its sides, toward camera 152. Alternatively to forming a continuous surface 210, the reflective surface may include a plurality of disjoint/physically separated reflective areas and/or the reflective surface may include one or more holes or slots which allow forward and backward viewing concurrently. Optionally, some of the reflecting areas or area is configured for reflecting light from a light source towards the imaged area. Optionally or alternatively, a light source (e.g., an optical fiber end or a LED) is integrated into a reflecting area and/or other backward pointing parts of the catheter.

Surface 210 optionally defines a mirror with a reflection area of at least 10 square millimeters, at least 20 square millimeters or even at least 30 square millimeters, to allow delivering of a relatively large image to imaging head 109. Optionally, part of surface 210 is aimed to reflect light from a light source to a viewing area, but not towards the imager.

As shown in FIGS. 3A and 3B, strings 172 and 174 (or wires, cables or other tension elements) running along, optionally within, handle 202 are used to fold and unfold reflector head 204. In some embodiments of the invention, strings 172 and 174 are manually manipulated by a device user. Alternatively, a mechanism (not shown) in handle 104 is used to manipulate the strings in order to fold and/or unfold reflector head 204, using methods known in the art.

Alternatively or additionally to strings 172 and 174, other mechanisms may be used to fold and/or unfold reflector head 204, such as electromagnets. In some embodiments of the invention, reflector head 204 includes springs which force head 204 into the open state when string 172 is released. Optionally, in these embodiments, string 174 is not included with reflector unit 200.

Alternatively to including mechanisms for both folding and unfolding, only a mechanism for unfolding reflector head 204 is provided, for simplicity. After use, reflector unit 200 is removed from the patient in its open state, together with insertion tube 102. In another exemplary embodiment of the invention, a biodegradable material holds reflector head 204 in its closed state during insertion to the patient. After insertion, the biodegradable material dissolves and reflector head unfolds. Alternatively or additionally, reflector head 204 comprises partially or entirely a biodegradable material which is dissolved to allow easier removal from the patient. Optionally, removal is via the digestive system. In some embodiments of the invention, reflector head 204 is expanded by injecting a saline solution at it (e.g., if it expands by absorbing fluid) and/or into it.

In some embodiments of the invention, segments 208 are rectangular. Optionally, in these embodiments segments 208 are all the same size. Alternatively, segments 208 may form together a triangle, or semi triangle (e.g., a rounded edge triangle), each segment having a trapezoid shape and/or different segments 208 having different sizes. In other embodiments of the invention, other shapes are used.

Surface 210 in the open state of reflector head 204 and handle 202 optionally define between them an angle greater than 90° or even greater than 100°, which directs images both from behind and from the side towards imaging head 109. Alternatively, any other angle is defined between surface 210 and handle 202, possibly an angle smaller than 90°. In some embodiments of the invention, the angle between surface 210 and handle 202 is fixed. Alternatively, the angle between surface 210 and handle 202 is adjustable, for example to vary the region being viewed and/or to move reflector head 204 out of the view of imaging head 109 or to aid in retraction of reflector head 204 into channel 120. In some embodiments of the invention, a pull wire extending through handle 202 is used to adjust the relative angle between surface 210 and handle 202 and/or the shape of surface 210.

In some embodiments of the invention, during a medical process, when it is desired to view areas beyond the distal end of insertion tube 102 and/or areas which reflector head 204 blocks, reflector head 204 is optionally collapsed. When a backward view is desired, reflector head 204 may be opened and a view of body regions behind camera 152 is directed by surface 210 toward the camera.

The opening and collapsing of reflector head 204 may optionally be performed a plurality of times during a single medical procedure, as required. For example, in advancing through an intestine, an insertion tube may be used to view a plurality of locations along the length of the intestine. In each location, reflector head 204 may be opened for inspection of the walls of the intestine, and collapsed in order to allow smooth movement of the insertion tube to another location.

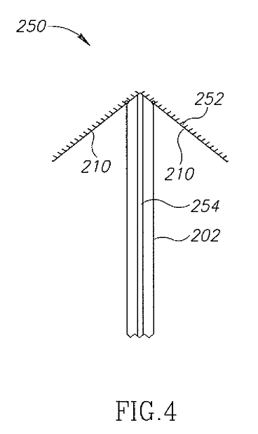

FIG. 4 is a schematic illustration of a reflector unit 250, in accordance with another exemplary embodiment of the invention. Reflector unit 250 comprises an umbrella shaped reflector head 252, having reflective surfaces 210, which can be opened and/or closed by a control staff 254 extending through a handle 202. As shown, reflector head 252 is symmetrical around handle 202. In some embodiments of the invention, however, reflector head 252 is longer on one side than on the other, for example when it is desired to pass through a working channel 120 not centralized in the cross section of insertion tube 102. Optionally, all of surfaces 210 are reflective. Alternatively, only one of surfaces 210 is reflective to reduce production costs.

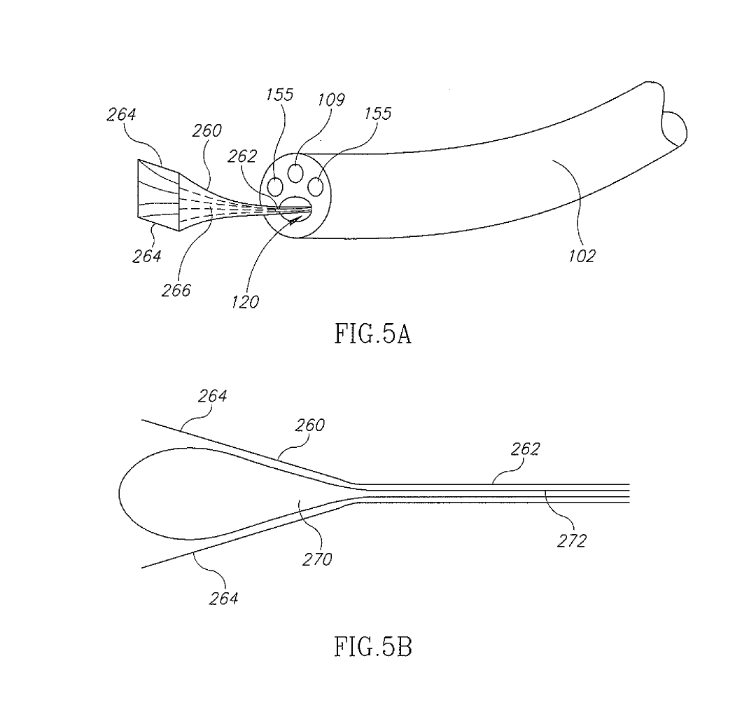

FIG. 5A is a schematic illustration of an insertion tube 102, with an expandable reflector head 260, in accordance with an exemplary embodiment of the invention. Insertion tube 102 optionally comprises an imaging head 109, one or more light sources 155 and a working channel 120. An elongate rod 262 carrying expandable reflector head 260 on its distal end, is adapted to pass through channel 120. In some embodiments of the invention, expandable reflector head 260 can only pass through channel 120 in its collapsed state. Alternatively, expandable reflector head 260 can pass through channel 120 also when it is partially or entirely open.

FIG. 5B is a schematic cross-sectional view of expandable reflector head 260, in accordance with an exemplary embodiment of the invention. Expandable reflector head 260 optionally comprises a pair of relatively rigid flat reflective surfaces 264 fixedly connected to each other at a proximal end and expandably connected in a manner which allows the flat surfaces to close to a state in which they are laid back to back against each other and to open to a state in which they are distanced from each other at their distal end, defining a pyramid shape. Optionally, flat surfaces 264 are connected to each other by elastic ribs 266 (FIG. 5A) which expand when the distal ends of surfaces 264 are distanced from each other. Alternatively or additionally, flat surfaces 264 are connected through folded ribs which open and close like an accordion. The use of relatively rigid surfaces allows for relatively accurate known image return from reflective surface 264, without distortions. In some embodiments of the invention, reflective surfaces 264 are elastic.

In some embodiments of the invention, reflector head 260 is at rest in a collapsed state, for example by one or more springs connecting surfaces 264 to each other and/or by it being formed from a shape-memory material. A balloon 270 is optionally inflated to apply force against surfaces 264 and thus expand reflector 260. Alternatively, reflector head 260 does not have a preset state. Reflector head 260 is optionally opened by balloon 270 and/or optionally closed by retracting it into channel 120 or by using any other appropriate method. Balloon 270 is optionally inflated through a feeding tube 272.

Further alternatively, reflector head 260 is at rest in an open state, for example using shape memory materials and is closed by pulling it into channel 120. As with the other embodiments described herein, this alternative may be used with substantially all the other embodiments of the present invention.

Alternatively to relatively rigid reflective surfaces, in some embodiments of the invention, reflector head 260 comprises a plurality of flexible surfaces which can be unfolded into flat surfaces. Optionally, the flexible surfaces are held by rigid ribs which are distanced from each other to make the flexible surfaces flat. In these embodiments, reflector head 260 optionally includes at least three, at least four or even at least five planar reflective surfaces. Alternatively, reflector head 260 may include only a single flat planar surface and the remaining walls are elastic or otherwise not planar. In some embodiments of the invention, one or more non-flat reflective surfaces may be used. Further alternatively or additionally, any other expansion structures may be used for reflector head 260, for example any of the structures used in umbrellas or for other intrabody expandable structures.

Alternatively to using flat reflective surfaces, in some embodiments of the invention, concave surfaces are used in order to achieve magnified images and/or convex surfaces are used in order to achieve a large view. In some embodiments of the invention, different reflective surfaces on different radial portions of a reflector head have different optical characteristics (e.g., have different surface geometries). For example, in one direction the reflective surface is flat, in a second direction it is concave and in a third direction it is convex. By rotating a handle of the reflector head, the specific reflective surface used is chosen. Optionally, such a structure is achieved using a balloon or membrane with internal struts that set the shape and/or elastically open to the shape.

### Collapsible Tube

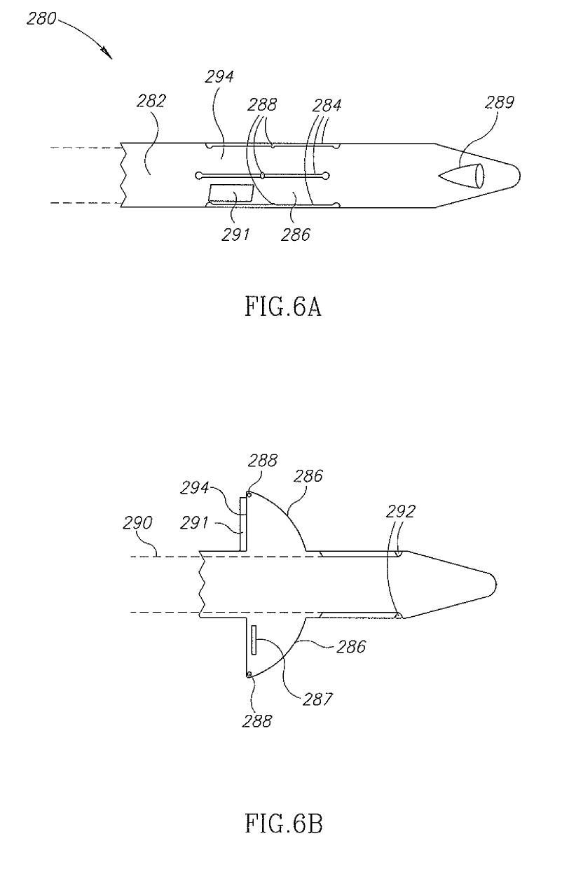

FIGS. 6A-6C are schematic illustrations of a reflector unit 280, in accordance with still another exemplary embodiment of the invention. Reflector unit 280 comprises a tube 282 sized to pass through a channel of insertion tube 102 (FIG. 2). A plurality of slits 284 in tube 282 optionally define bendable strips 286. Preferred bend points 288 are optionally defined in strips 286, such that upon applying a proximally directed force on tube 282, strips 286 bend to the state shown in FIG. 6B. Bend points 288 are optionally rounded in order to relieve stress and prevent cracking. In an exemplary embodiment of the invention, the proximally directed force is applied by pulling on an inner actuating tube 290 connected to tube 282 at one or more points 292 distal of strips 286. Inner actuating tube 290 may optionally also be used also to straighten strips 286, for example when desired to retract reflector unit 280 through the channel. Alternatively or additionally to inner actuating tube 290, other tension elements (e.g., wires, pushers) may be used to actuate the proximally directed force. Alternatively to requiring a pulling force to be opened, strips 288 may be designed to bend when they exit a working channel of the endoscope.

One or more of proximal portions 294 of bendable strips 286, proximal to bend points 288, are coated with a reflective material or formed from a reflective material, such that these one or more proximal portions 294 serve as mirrors for directing images toward imaging head 109. Alternatively or additionally, a mirror 291 is mounted on one or more of proximal portions 294. Further alternatively or additionally, one or more of proximal portions 294 are transparent, and a mirror 287 is positioned behind them. Optionally, the extent of bending of strips 286 is adjustable by a user of the device, for example by the extent of pulling on inner tube 290, such that proximal portions 294 may be positioned at an angle of 90° relative to tube 282, as shown in FIG. 6B, or at greater or smaller angles.

In some embodiments of the invention, tube 282 includes three bendable strips 286 and proximal portions 294, as shown in FIG. 6C. Alternatively, tube 282 includes more than three strips 286 or fewer than three strips. In some embodiments of the invention, the reflective surfaces of all the strips 286 bend together to the same angle. Alternatively, different reflector surfaces of different strips 286 have different angles with tube 282 at a given extent of pulling on tube 290. Alternatively or additionally to all of strips 286 defining flat reflective surfaces, one or more of the defined reflective surfaces may be convex or concave to achieve a wider view or larger images.

In an exemplary embodiment of the invention, tube 282 has a diameter of about 3.5 millimeters and each of three strips 286 has a width of about 3.6 millimeters. Other dimensions may also be used, for example in which slits 284 are wider and cover at least 10% or even more of the circumference of tube 282.

Optionally, reflector unit 280 serves solely for directing images at imaging head 109. Alternatively, reflector unit 280 may carry additional apparatus, such as a camera 289 (FIG. 6A) and/or an anchoring balloon (as discussed below). In some embodiments of the invention, reflector surfaces in accordance with embodiments of the present invention may be used additionally to direct light towards tissue and/or to shade tissue from light of the endoscope.

FIG. 6D is an alternative implementation of a foldable tube 296, in accordance with an exemplary embodiment of the invention. Foldable tube 296 comprises an elastic braid 298, formed from a super elastic alloy or a plastic, instead of slits 284 and strips 286. Elastic braid 298 is foldable in manner similar to strips 286, to the orientation shown in FIG. 6B. One or more reflective surfaces 299 mounted on a proximal portion of the braid serves as a mirror for directing images toward camera 152.

Tubes 282 and 296, as well as the other embodiments of reflective heads, are optionally rotatable within the patient, to allow freedom in directing images by the reflective surfaces toward imaging head 109.

### Balloon Reflector

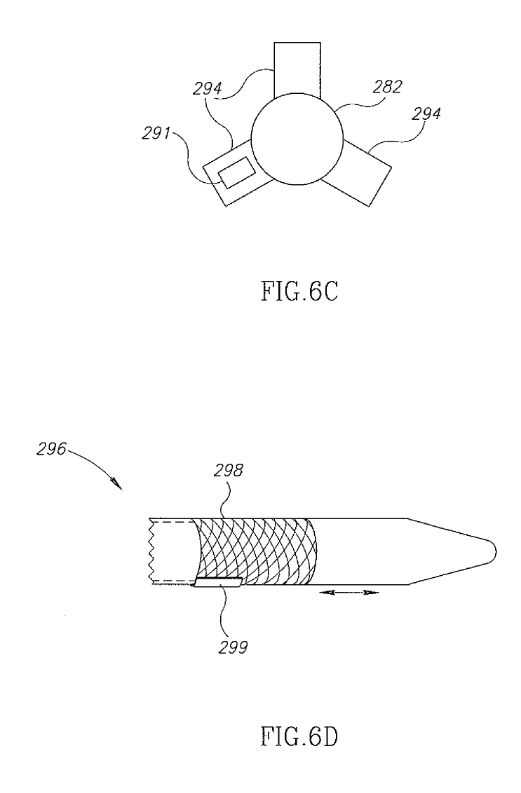

FIG. 7 is a schematic cross sectional illustration of a reflector unit 300, in accordance with still another exemplary embodiment of the invention. Reflector unit 300 comprises a handle (or shaft) 202 and a balloon 302 mounted at its distal end. Part or all of the outer surface of balloon 302 comprises a reflective material or is coated by a light reflective coating, forming a reflective surface 304. Alternatively, a flexible mirror is attached to the balloon. Optionally, the attached mirror tends to be planar, for example by being formed from a super-elastic material with that shape.

In some embodiments of the invention, reflective surface 304 covers less than 30%, 20% or even less than 10% of the outer surface of the balloon. Alternatively, reflective surface 304 covers more than 30%, more than 50% or even more than 70% of the outer surface of the balloon. In some embodiments of the invention, at least the proximal half of the balloon is, mostly or substantially entirely, reflective. Optionally, portions of the balloon which are not reflective are made absorptive to prevent them from reflecting light in a manner which interfere with the imaging.

Optionally, balloon 302 comprises a relatively rigid plastic, such as Nylon, polycarbonate, acrylic, PET or PETG. Alternatively or additionally, balloon 302 comprises a non-rigid plastic, such as polyethylene, polyurethane or PVC. In some embodiments of the invention, reflective surface 304 comprises Mylar or another biaxially oriented PET film, a deposited or adhered metal layer, such as vacuum aluminum, gold and/or platinum (optionally with a pre-coating of titanium). Optionally, the image is color corrected by processing for any color effect of the reflective coating and/or a suitable light source chromaticity is selected.

In accordance with this embodiment, reflective surface 304 changes its optical characteristics (e.g., curvature) in moving between its collapsed state and its open state. In some embodiments of the invention, while viewing images using reflective surface 304, a device user can adjust the surface geometry of the reflective surface, for example its convexity, in order to close in on a feature of interest or to expand the view around a feature of interest.

Optionally, balloon 302 has a preferred inflated shape, for example in which reflective surface 304 is planar, so that it does not distort images it reflects toward camera 152 (FIG. 2). Alternatively, reflective surface 304 defines a non-flat (e.g., parabolic, spherical, convex) mirror, which provides a view of a relatively large area to camera 152. Further alternatively, reflective surface 304 defines a concave surface, for example by using a super-elastic metal layer on the outer surface of the balloon. In an exemplary embodiment of the invention, balloon 302 is radially asymmetric, having different surface shapes in different directions. In some embodiments of the invention, the preferred expanded shape is achieved by including stiffening ribs 308 in the balloon, in a manner which creates the preferred expanded shape when the balloon is expanded. Alternatively or additionally, the balloon is made of different materials and/or displays different thicknesses or densities in different areas.

In some embodiments of the invention, control station 110 includes a correction software adapted to correct acquired images for distortions due to the shape of reflective surface 304. Optionally, in the preferred expanded shape, reflective surface 304 distorts images in a known manner for which the correction software of control station 110 is calibrated. Alternatively or additionally, insertion tube 102 has on its distal end a unique image used in calibrating the software. When balloon 302 is inflated, it is first used to direct the unique image to camera 152, for calibration. Thereafter, images acquired via reflective surface 304 are corrected in a similar manner. Alternatively or additionally, the reflective surface has one or more lines or other patterns marked on it, which are used by the correction software to identify that it is acquiring images reflected by the reflective surface and/or to determine the distortion caused by the reflective surface. Further alternatively or additionally, the light source of the endoscope projects a predetermined pattern which is identified in its distorted form by the correction software.

In some embodiments of the invention, the correction software compensates for and/or identifies holes and/or disjoint areas in the reflective surface, for example separating into separate images portions of the image acquired through the reflective surface and portions acquired through a hole or slot in the reflective surface.

While in some embodiments of the invention the reflective surface provides an image with a substantially same resolution along its entire area, in other embodiments of the invention the provided images have different resolution in different areas. In some embodiments of the invention, the correction software indicates on displayed images the quality level of the displayed regions. Alternatively or additionally, the reflective software includes lines or other patterns that identify high or low quality regions.

In some embodiments of the invention, the preferred expanded shape is selected according to the organ in which the endoscope is used. For example, in narrow organs, such as the intestine, a first expanded shape is used, while in larger organs, such as the stomach, a second expanded shape is used. In the nasal passage or esophagus, a relatively limited expansion may be preferred, while in the bladder a larger expansion may be preferred.

Optionally, balloon 302 is inflated to a predetermined volume and/or pressure at which the preferred expanded shape is achieved. In some embodiments of the invention, balloon 302 has a plurality of predetermined expanded shapes for which the correction software is calibrated. Optionally, one or more first shapes are flat or convex, while when more inflated the reflective surfaces become flat and/or concave, so that a device user can select desired optical properties according to the extent of inflating of the balloon. Optionally, the shape is initially shaped by stiffening ribs and the stiffening ribs can be distorted by inflation of the balloon. Alternatively or additionally, the specific correction to be applied to the acquired images is determined based on the inflation extent and/or pressure of balloon 302. In some embodiments of the invention, the inflation extent of balloon 302 is determined using an external imaging method such as X-ray imaging. Alternatively or additionally, position sensors 306 within balloon 302 report the layout of reflective surface 304.

### Further Embodiments

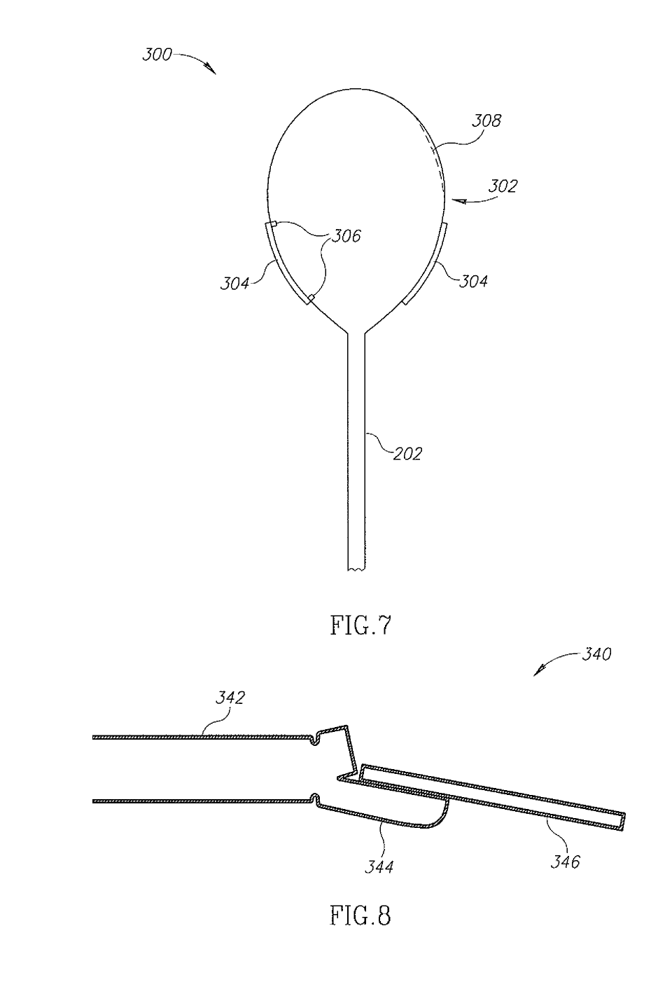

FIG. 8 is a schematic cross-section illustration of a reflector unit 340, in accordance with another exemplary embodiment of the invention. Reflector unit 340 comprises a handle 342 similar to that used in jaw tools, such as described in US patent publication 2005/0251166, to Vaughan et al., published Nov. 10, 2005, the disclosure of which is incorporated herein by reference, or of any other type known in the art, optionally, with only one jaw 344 and optionally a partial second jaw to allow opening and closing of the jaws, without the partial jaw interfering. A reflective surface 346, such as a stainless steel sheet, is mounted on jaw 344, such that movement of the jaw (e.g., opening and closing) changes the angle of reflective surface 346 between an axial state in which reflective surface 346 can pass through a channel of insertion tube 102 and a reflection state in which reflective surface 346 is positioned in a manner which allows it to direct images toward camera 152. Reflective surface 346 optionally has a small enough width allowing it to pass in its axial state through a channel of insertion tube 102. In an exemplary embodiment of the invention, reflective surface 346 is a polished stainless steel sheet of 3.5×7 millimeters. Alternatively or additionally, reflective surface 346 is expandable and/or unfoldable sideways.

In an exemplary embodiment of the invention, reflector unit 344 is manufactured from a jaw tool from which one of the jaws is removed or merely ignored. Optionally, reflector unit 344 is welded or glue bonded onto the jaw, although any other suitable attachment method may be used. Alternatively or additionally, reflector unit 344 is formed with an elastic sleeve or other band which is mounted on the jaw.

The orientation of reflective surface 346 is optionally controllable in a manner similar to the control of jaw tools, for example using steel pull wires.

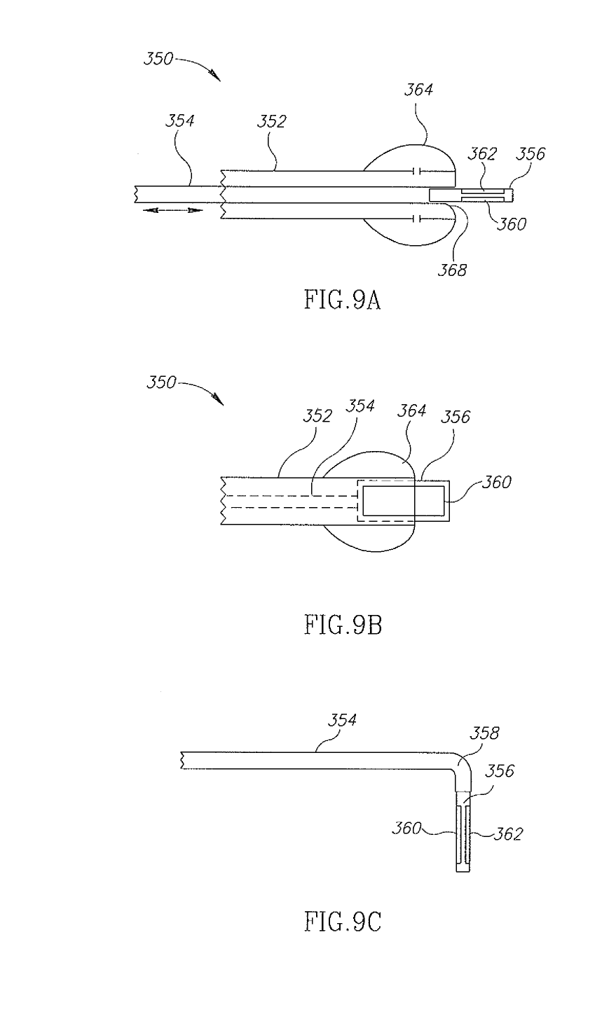

FIGS. 9A and 9B are side and back cross-section views of a reflection unit 350, in accordance with an exemplary embodiment of the invention. Reflection unit 350 comprises an outer tube 352 and an inner tube 354, which is axially movable (e.g., slideable, screwable) relative to outer tube 352. At its distal end, inner tube 354 carries a reflective sheet 356 which can be held within outer tube 352 and extended out of outer tube 352. Alternatively or additionally to being reflective, sheet 356 carries a rear view mirror 360. In some embodiments of the invention, sheet 356 also carries a forward view camera 362.

FIG. 9C illustrates inner tube 354 with reflective sheet 356 in a rest state, in accordance with an exemplary embodiment of the invention. A distal portion of inner tube 354 optionally comprises a shape memory alloy wire or other elastic material. In its rest state, inner tube 354 optionally has a 90° bend 358 at a point close to the connection with reflective sheet 356. When inner tube 354 is retracted into outer tube 352, reflective sheet 356 axially extends with outer tube 352, such that imaging unit 350 has a small cross section, for example with a diameter of less than 3.5 millimeters, so that imaging unit 350 may pass through a working channel of an endoscope. When, however, inner tube 354 is extended out of outer tube 352 (FIG. 9A), mirror 360 and optional camera 362 are deflected to the rest state of FIG. 9C to acquire images. It is noted that inner tube 354 may be only partially extended out of outer tube 352, in which case mirror 360 is at a smaller angle relative to outer tube 352 than in the rest state, so as to direct mirror 360 at any of a large span of angles. Outer tube 352 optionally has a rounded distal edge 368, allowing smooth movement of inner tube 354 relative to the outer tube.

In some embodiments of the invention, outer tube 352 carries a balloon 364 (FIG. 9B) which may be inflated through the interior of tube 352 or a channel passing therethrough. The inflating of balloon 364 may be used to anchor reflection unit 350 within the patient, for example when reflection unit 350 additionally serves as a guidewire. During insertion, balloon 364 is deflated to allow fast insertion. When a desired location is reached, balloon 364 is optionally inflated to achieve anchoring of unit 350 in place.

It is noted that a balloon may be mounted on any of the reflection units described above. The balloon optionally comprises an inflated disc shaped balloon which expands radially from the outer surface of outer tube 352.

In the open states of the reflector heads of some of the above described embodiments, the reflector is optionally sufficiently smooth to reflect light at sufficient quality to pass a view to the imaging unit at sufficient quality to allow medical analysis of the images. In some embodiments of the invention, the images acquired through the reflection surface undergo a resolution reduction, after correction (if performed) of less than a factor of 4, less than a factor of 2 and optionally less than a factor of 1.5.

FIGS. 1A-10C show schematic cross-sectional illustrations of reflector units 300, in accordance with still additional exemplary embodiments of the invention. A reflector unit 300 comprises a handle (or shaft) 202 and a balloon 302 mounted at its distal end. Part or all of the outer surface of balloon 302 comprises a reflective material or is coated by a light reflective coating, forming a reflective surface 304. Alternatively, a flexible mirror is attached to the balloon (not shown). Optionally, the attached mirror tends to be planar, for example by being formed from a planar super-elastic material.

In some embodiments of the invention, reflective surface 304 covers less than 30%, 20% or even less than 10% of the outer surface of the balloon. Alternatively, reflective surface 304 covers more than 30%, more than 50% or even more than 70% of the outer surface of the balloon. In some embodiments of the invention, at least the proximal half of the balloon is, mostly or substantially entirely, reflective. Optionally, portions of the balloon which are not reflective are made absorptive to prevent them from returning light which can interfere in the imaging. Optionally a masking is used when depositing or coating with a reflective layer. Alternatively, the entire balloon is made reflective. Optionally, a part that is not desired to be reflective is then coated with an absorbing layer.

Optionally, balloon 302 comprises a relatively rigid plastic, such as Nylon, polycarbonate, acrylic, PET or PETG, optionally folded in pleats. Alternatively or additionally, balloon 302 comprises a non-rigid plastic, such as polyethylene, polyurethane or PVC. In some embodiments of the invention, reflective surface 304 comprises Mylar or a deposited metal layer.

In accordance with this embodiment, reflective surface 304 optionally changes its optical characteristics (e.g., curvature) in moving between its collapsed state and its open state. In some embodiments of the invention, while viewing images using reflective surface 304, a device user can adjust the surface geometry of the reflective surface, for example its convexity, in order to close in on a feature of interest and/or to expand the view around a feature of interest.

Optionally, balloon 302 has a preferred inflated shape, for example in which reflective surface 304 is planar, so that it does not distort images it reflects toward camera 152 (FIG. 2). In an exemplary embodiment of the invention, balloon 302 is generally trapezoidal, with the angle between handle (202) and reflective surface (304) being 135 degrees or greater or smaller angles. In some embodiments of the invention, the preferred expanded shape is achieved by including stiffening ribs (not shown) in the balloon, in a manner which creates the preferred expanded shape when the balloon is expanded. Alternatively or additionally, the balloon is made of different materials and/or displays different thicknesses or densities in different areas.

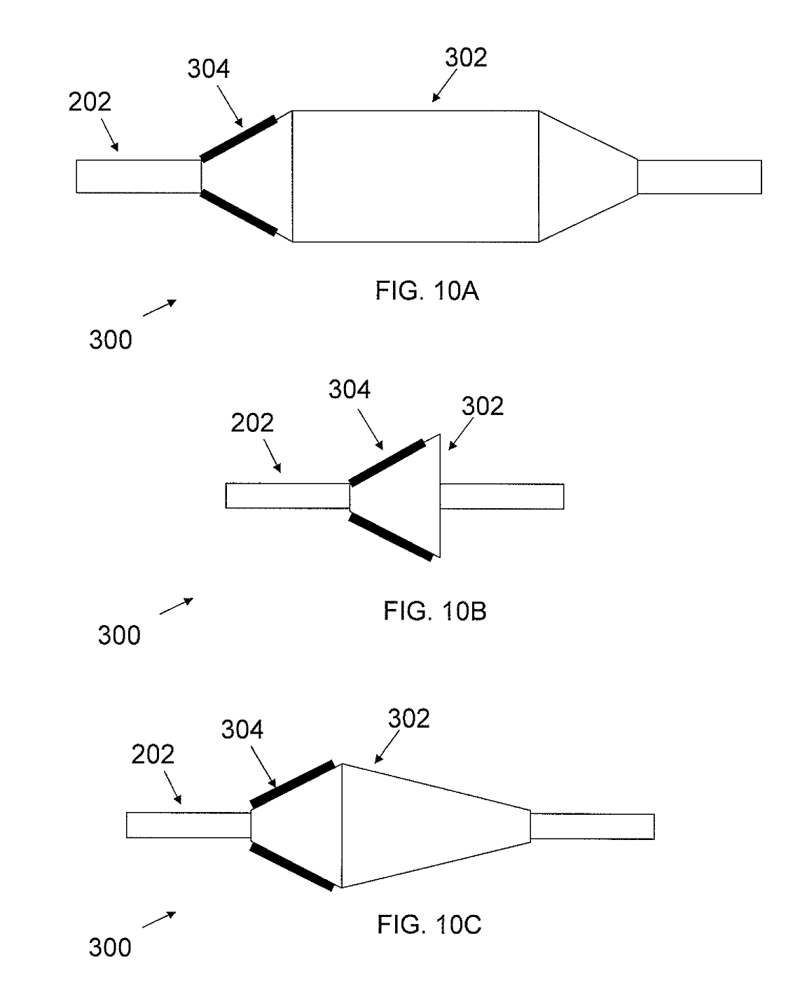

Referring specifically to FIG. 10A, the balloon (302) includes a cylindrical section and a tapered reflector and a tapered distal side. Optionally, this cylindrical section aids in providing a relatively planar reflection portion.

Referring specifically to FIG. 10B, the non-reflecting portion of the balloon is minimized, for example, with the balloon having a triangular or trapezoid cross-section, with only a proximal taper. This may reduce interference with movement of the endoscope. This may also be advantageous in anatomy where there is limited space for a larger balloon.

Referring specifically to FIG. 10C, it may be easier to manufacture a form with no cylindrical portion and where the distal end of the balloon is tapered, optionally in a manner symmetric with the reflecting (proximal) side, alternatively more or less tapered. Optionally, the tapering is selected for assistance in advancing of the balloon, for example, for changing a center of field of view and/or a size of a field of view. The tapered shape may also aid in the folding of a deflated balloon, and/or result in a smaller diameter of the folded, deflated balloon. Optionally or alternatively, the taper is selected so as to allow the balloon to be more easily drawn into the endoscope channel after it is deflated.

In some embodiments, the imager is backwards looking and the reflecting element is mounted on the body of the endoscope proximal to the imager and serves to selectively reflect a forward image. Optionally, the imaging modality is non-optical, for example, ultrasonic, with a suitable ultrasonic reflector being used.

In some embodiments of the invention, control station 110 includes a correction software adapted to correct acquired images for distortions due to the shape of reflective surface 304. Optionally, in one or more expanded states, reflective surface 304 distorts images in a known manner for which the correction software of control station 110 is calibrated. Alternatively or additionally, insertion tube 102 has on its distal end a unique image used in calibrating the software. When balloon 302 is inflated, it is first used to direct the unique image to camera 152, for calibration. Thereafter, images acquired via reflective surface 304 are corrected in a similar manner. Alternatively or additionally, the reflective surface has one or more lines or other patterns marked on it, which are used by the correction software to identify that it is acquiring images reflected by the reflective surface and/or to determine the distortion caused by the reflective surface. Further alternatively or additionally, the light source of the endoscope projects a predetermined pattern which is identified in its distorted form by the correction software.

### Small Reflector

Alternatively to using an expandable reflector head, a reflector head including a single segment, which passes through working channel 120, is used. In accordance with this alternative, handle 202 optionally has sufficient maneuverability to allow positioning of reflector head 204 close to imaging head 109, to allow for imaging of a sufficiently large region and/or a curved mirror is used. Optionally, the single segment is preconfigured to bend out of the axis of the working channel.

It will be appreciated that the above-described methods may be varied in many ways, including but not limited to changing materials, sizes and shapes. For example, in some embodiments of the invention, imaging head 109 and insertion tube 102 do not include a lens. The above described methods and apparatus may be used with substantially any type of endoscope, including disposable and non-disposable endoscopes, endoscopes with or without covering protective sheaths and/or with or without separate control handles. In some embodiments of the invention, a reflector head or reflection unit in accordance with the above described embodiments is used with the endoscope described in U.S. provisional patent application 60/806,162, filed Jun. 29, 2006 or with any of the endoscopes described in U.S. provisional patent application 60/763,267, filed Jan. 30, 2006, the disclosures of which are incorporated herein by reference.

In some embodiments of the invention, the reflector head (e.g., 204) is made from inexpensive materials, so that it is disposable after every medical procedure. Alternatively, the reflector head is made with smooth surfaces which allow fast and simple sterilization, for example using gas or heat or steam.

It should also be appreciated that the above described description of methods and apparatus are to be interpreted as including apparatus for carrying out the methods, and methods of using the apparatus.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art. Furthermore, the terms “comprise,” “include,” “have” and their conjugates, shall mean, when used in the claims, “including but not necessarily limited to”.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims.

1. An endoscopic system, comprising:
an insertion tube, defining at least one channel along its length;
an imaging unit on the insertion tube; and
a reflector adapted to direct a view at the imaging unit and adapted to pass through the at least one channel and extend out from the end of the insertion tube.

2. An endoscopic system according to claim 1, wherein the reflector is mounted on a collapsible frame having an open state in which the reflector cannot pass through the at least one channel and a collapsed state in which the reflector can pass through the at least one channel.

3. An endoscopic system according to claim 1, wherein the reflector is mounted on a deflectable handle, deflected between a state in which the reflector cannot pass through the channel and a state in which the reflector can pass through the channel.

4. An endoscopic system according claim 1, wherein the reflector is mounted on a frame including a plurality of arms having a fixed relative orientation at one end and adapted to move relative to each other on another end.

5. An endoscopic system according to claim 1, wherein the reflector is a collapsible reflector having an open state in which it is larger than the cross-section of the channel and a collapsed state in which it can pass through the channel

6. An endoscopic system according to claim 5, wherein the reflector is adapted to move a plurality of times between the open and collapsed state.

7. An endoscopic system according to claim 5, wherein the reflector is adapted to be folded in changing between the open state and the collapsed state.

8. An endoscopic system according to claim 1, wherein the reflector is adapted to change an optical curvature thereof in changing between the collapsed state and the open state.

9. An endoscopic system according to claim 1, wherein the reflector is mounted on a balloon or is part of a balloon.

10. (canceled)

11. An endoscopic system according to claim 9, wherein the balloon is adapted to be inflated to a state in which a reflective surface thereof is flat or piecewise flat.

12. An endoscopic system according to claim 9, wherein the balloon is reinforced by stiffening ribs.

13. An endoscopic system according to claim 9, wherein the balloon is adapted to have a predetermined shape when inflated.

14. An endoscopic system according to claim 1, wherein the reflector is adapted to have a parabolic or spherical surface over most of an area that directs light toward the imaging unit.

15. An endoscopic system according to claim 1, wherein the reflector is adapted to have a convex surface over most of its area directing light toward the imaging unit.

16. An endoscopic system according to claim 1, wherein the imaging unit is at the distal end of the insertion tube.

17. (canceled)

18. An endoscopic system according to claim 1, comprising a correction software which corrects images for distortions caused by the reflector.

19. An endoscopic system according to claim 1, wherein the reflector in an unexpanded form is adapted to pass through a channel having a diameter of less than 4 millimeters.

20. An endoscopic system according to claim 1, wherein the reflector is adapted to be positioned relative to the imaging unit such that it directs a view from behind the imaging unit at the imaging unit.

21. (canceled)

22. An endoscopic system according to claim 1, wherein the reflector is adapted to have a planar surface over most of an area of the reflector that is adapted to direct light toward the imaging unit.

23. A method of viewing images within a patient comprising:
inserting an endoscope into the patient;
providing a reflector within the endoscope;
moving the reflector outside the endoscope; and
acquiring images of tissue of the patient reflected by the reflector.

24. A method according to claim 23, comprising maintaining the reflector in collapsed form while it is within the endoscope, moving the reflector out a channel of the endoscope, and uncollapsing the reflector after it is moved out of the endoscope.

25. A method according to claim 24, wherein the reflector is mounted on a collapsible frame and comprising uncollapsing the frame after the reflector is moved out of the distal end of a channel of the endoscope.

26. A method according to claim 23, wherein the reflector is mounted on a handle, the method comprising deflecting the handle after the reflector is moved out of the distal end of a channel of the endoscope.

27. The method of claim 23, further comprising moving the reflector back into a channel of the endoscope after acquiring images.

28. The method of claim 23, further comprising removing the reflector from the patient through the channel, while the endoscope is within the patient.

29. The method of claim 23, wherein providing a reflector along the endoscope comprises passing the reflector along an outer surface of the endoscope.

30. The method of claim 23, wherein providing a reflector along the endoscope comprises passing the reflector along a channel extending through the endoscope.

31. A method according to claim 30, wherein the reflector is elastically loaded in the channel, such that it expands upon exiting a distal end of the channel.

32. A method according to claim 31, comprising retracting the reflector into the channel in a manner which causes the reflector to collapse due to the retraction.

33. The method of claim 23, further comprising changing a geometry of the reflector while it is within the patient.

34. An endoscopic system, comprising:
an insertion tube;
an imaging unit on the insertion tube; and
a reflector unit including a reflector and having a collapsed state, and an open state, wherein the reflector changes its geometry between the open and collapsed states of the reflector unit, and wherein the reflector is adapted to direct a view from behind the imaging unit at the imaging unit when in open state.

35. An endoscopic system according to claim 34, wherein the reflector unit comprises a balloon having a reflective surface.

36-37. (canceled)

38. An endoscopic image reflecting unit, comprising:
an elongate handle adapted to pass through a channel of an endoscope; and
a collapsible reflector head carrying a reflector having a collapsed state in which it is adapted to pass through a channel of an endoscope, and an open state in which it is suitable to direct images toward an imaging unit at sufficient quality for imaging.

39. A unit according to claim 38, wherein the collapsible reflector head comprises a distal part of the handle adapted to be bent into a state in which the reflector is at an angle of at least 45 degrees with an axial axis of the handle and to be bent into an axial state in which a length of the reflector substantially coincides with the axial axis of the handle.

40. A unit according to claim 39, wherein the collapsible reflector head has a single rest state, and wherein in the single rest state the reflector is at an angle of at least 45 degrees with an axial axis of the handle.
